(19) United States
(12) Patent Application Publication
(10) Pub. No.: US 2021/0283043 A1
(43) Pub. Date: Sep. 16, 2021

(54) COCOA BUTTER COMPOSITION, AND PROCESS FOR OBTAINING SAME

(71) Applicant: CORMAN S.A., Limbourg (BE)

(72) Inventors: Barbara LEPILLEUR, Jalhay (BE); Marleen VANRUSSELT, Diest (BE); Daniel DALEMANS, Herstal (BE)

(73) Assignee: CORMAN S.A., Limbourg (BE)

(21) Appl. No.: 17/259,042

(22) PCT Filed: Jul. 11, 2019
(86) PCT No.: PCT/EP2019/068697

§ 371 Date: Jan. 8, 2021

(30) Foreign Application Priority Data
Jul. 11, 2018 (BE) BE2018/5486

(51) Int. Cl. A61K 8/92; A23D 7/00; A23D 7/04; A23G 1/36; A23G 9/32; A23G 3/40; B01D 3/12; B01D 3/14; A61K 8/02; A61Q 19/00

(52) U.S. Cl. CPC A61K 8/922; A23D 7/001; A23D 7/04; A23G 1/36; A23G 9/327; A23G 3/40; A61K 2800/10; B01D 3/143; A61K 8/0216; A61Q 19/00; A23V 2002/00; A61K 2800/24; B01D 3/12

(57) ABSTRACT

The invention relates to a new cocoa butter composition, to a new process for obtaining same by one or more fractional molecular distillation procedures, and to the specific fractions obtained.

### TECHNICAL FIELD OF THE INVENTION

The present invention lies in the agri-food field and relates to a new cocoa butter composition and to a process for obtaining a cocoa butter, more specifically to the process for obtaining the said cocoa butter composition, by one or more steps of fractional molecular distillation, either alone or in combination with other fractionation processes. The present invention also relates to food or non-food compositions that incorporate this cocoa butter composition of the invention.

### Technological Contextual Background Upon which the Invention is Based

Cocoa butter is the fat obtained from the cocoa bean of Theobroma cacao (designating all cocoa trees) and is defined as an edible fat.

Cocoa butter has a composition and physical-chemical properties that are specific to it which further make this fat unique. (Reference for description of the composition of the cocoa butter based on various different origins, see: M. Lipp and all, 2001, Journal of food composition and analysis 14, 399-408).

In fact, cocoa butter is solid at ambient temperature thanks to its high melting point (around 35° C.) and melts very quickly. This behaviour of cocoa butter is of great interest in certain food industry sectors, such as the manufacture of chocolate. However, for other applications such as fillings, ice creams, etc for which the fat used must at ambient temperature, present a less hard or firm texture, similar to ointment-like or liquid-like texture, cocoa butter may not be used by itself or must be mixed with another plant-based or animal fat, in order to exhibit the required texture.

However, use of these types of mixtures precludes use of the descriptive nomenclature “pure cocoa butter” and therefore in order to retain use of the European descriptive labelling of “Pure Cocoa Butter Chocolate”, the latter may not be incorporated into the preparation of a confectionery product, such as chocolate bars or in ‘couverture chocolate’.

A modified cocoa butter composition is of great interest for various food industries, such as the Chocolate industry or manufacture of spreads, confectionery products, biscuits and cookies, UHT (ultra high temperature processing) and fresh desserts, etc, in order to adapt the texture of chocolate or chocolate-based products, without adding other types of fats of animal or plant origin (in particular in order to avoid using palm oil or shea oil) and therefore so as to retain use of the European descriptive labelling of “Pure Cocoa Butter Chocolate”.

A modified cocoa butter composition may also find non-food applications, for example in cosmetics, for the moisturising, anti-oxidant, soothing, regenerating or healing properties.

The fat of whole and healthy beans, that have been fermented and dried in a timely manner, stored in an appropriate manner and exported from their area of origin, shall generally have a content of Free Fatty Acids (FFA) of less than 1% and certainly less than 1.7%, calculated in relation to the weight of the bean which is the legal limit for cocoa butter in the European Union (directive 2000/36/EC) and in accordance with the standard on cocoa butter (86-1981, REV.1-2001) of the Codex Alimentarius of 2001.

Different fractionation processes well known to the person skilled in the art make it possible to separate the “liquid” and “solid” fractions of the fats.

In the case of cocoa butter, a high value-added fat, these fractionation by fractional crystallisation processes with or without the addition of a solvent (as described in the U.S. Pat. No. 5,069,915) and whether or not in combination with reactions of chemical or enzymatic modifications by inter-esterification (as described in the patent application WO2010/149323) seem costly and/or difficult to implement, on account of the particular cocoa butter composition.

Molecular distillation is a continuous process of physical fractionation of the constituents of a complex mixture. The principle thereof is based on the differences in vapour pressure of these constituents at a given temperature with the result that the lighter constituents have a higher vapour pressure and therefore get vaporised before the heavier constituents.

The operation is carried out at high temperature (greater than 200° C., or even 250° C.), for a very short time period (a few seconds) and under very high vacuum conditions (0.001 to 0.004 mbar, or about one millionth of atmospheric pressure). Then, continuous wiping of the product in contact with the vaporisation surface makes it possible to produce a thin film with constant agitation, which promotes heat exchange and evaporation of the desired constituents. The distance as well as the reduced time between the vaporisation zone and the condensation zone provide the ability to effectively carry out very efficient separations of compounds that nevertheless have close vapour pressures, and to maintain the high vacuum conditions required. Owing to this characteristic feature it is thus justifiable for the terminology for this technology, namely “short path distillation” also to be used to characterise the said process and for the evaporated fraction(s) to be referred to as “distillate(s)” or “olein (s)” and for the residual fraction(s) to be referred to as “residue(s)”, “retentate(s)” or “stearin(s)”.

### STATE OF THE ART

Molecular distillation is the most efficient process of fractionation by fractional evaporation. Thanks to this efficiency, molecular distillation makes it possible to carry out separations of constituents at a fairly low temperature, which ensures the preservation of the best quality of the fractions collected.

Molecular distillation and use thereof for fractionation of milk fat have been studied by Campos R. J et al (2003, Journal of Dairy Science, 86, 735-745.—Arul, J. et al) and show an advantage relating to reducing the levels of saturated fatty acids in the milk fat, as described in the patent application WO2009/05649.

However, this method of separation by molecular distillation has only been applied to cocoa butters that have been previously modified by major chemical treatment processes prior to any molecular distillation step. In these cases, the raw material subjected to molecular distillation is no longer able to satisfy the definition of a natural and untreated cocoa butter.

The patent application FR 2180242 describes a process of molecular distillation of a cocoa butter that is not refined and chemically treated after extraction of the bean, in order to obtain a concentrate of unsaponifiable matter and incorporating hydrocarbons, Tocopherols, terpene alcohols and sterols, for the use thereof as a cosmetic emulsion. The chemical treatment process necessary so as to obtain the raw material for this molecular distillation requires the use of chemical products and adds steps to the fractionation process for fractionating a cocoa butter.

The article ‘Optimization of deacidification of low-calorie cocoa butter by molecular distillation’ (WeiLiang et al, LWT—Food Science and Technology 46 (2012) 563-570) describes a process of molecular distillation of a trans-esterified cocoa butter, comprising about 23% of Gamma-linolenic Acid (GLA). The chemical treatment of this raw material subjected to the molecular distillation requires the use of chemical products and adds steps to the fractionation process for fractionating a cocoa butter. In addition, this trans-esterification increases the percentage of GLA in the raw material intended to undergo molecular distillation.

The thesis ‘Changing the functionality of cocoa butter’ (De Clercq N., Ghent University, 2012, ISBN 978-90-5989-470-9) describes a process of molecular distillation of a processed cocoa butter (enzymatic glycerolysis), that comprises approximately 4% AGL. The chemical treatment of the raw material subjected to molecular distillation requires the use of chemical products and adds steps to the fractionation process for fractionating the raw material.

### OBJECTS OF THE INVENTION

The object of the present invention is to obtain an improved cocoa butter composition that incorporates a higher percentage of certain lipids than a composition of an unprocessed cocoa butter, so as to ensure it does not have the disadvantages of the state of the art.

A preferred object of the present invention is to obtain such a composition which comprises fewer hard/firm fractions than a composition of a natural cocoa butter that is unprocessed but refined, from which it is preferably derived and which can thus find new advantageous applications in the food sector without modifying the taste of the food products obtained, in particular in the preparation of spreads, ice creams, couverture chocolate, fillings, or other food products or in the non-food sector, in particular in cosmetics.

Another object of the present invention is to obtain such a composition which continues to be considered to be “pure cocoa butter” and therefore may be incorporated into a food composition which, at the level and on the basis of European regulations, continues to be referred to as “chocolate”, in particular “Pure Cocoa Butter Chocolate”.

Another object of the present invention is to provide a production process for producing a cocoa butter composition, preferably a process for producing the cocoa butter composition of the invention, in particular a process which can be easily automated and can be industrialised for the production of large volumes of this novel cocoa butter composition of the invention.

A final object of the invention is to provide a treatment process for treating cocoa butter by means of a process which will make it possible to obtain a number of fractions which may all be recovered and exploited in the food sector, in particular for the preparation of a chocolate.

### SUMMARY OF THE INVENTION

A first aspect of the present invention relates to a cocoa butter composition, preferably of one or more cocoa butter distillate(s), having a composition comprising a particular percentage of lipids, also referred to as new lipid fractions, as well as a process for obtaining this new cocoa butter composition.

The cocoa butter composition is characterised by its high percentage of palmitic fatty acid (C16: 0) in relation to the composition of total fatty acids of an untreated, but refined cocoa butter, in particular the percentage by weight of palmitic acid (referred to as type C16:0) is greater than 27%, preferably greater than 27.5%; than 28%; than 28.5; than 29%; than 29.5%; than 30%; than 30.5%; than 31%; than 31.5%; than 32%; than 32.5% or than approximately 33%, these percentages by weight being calculated as an absolute value across the totality of the fatty acids in the composition of the cocoa butter of the invention, preferably of the cocoa butter distillate. Initially, the percentage of palmitic fatty acid in an untreated, but refined cocoa butter is approximately 24%.

In addition, the composition of the invention is also characterised by its low percentage of stearic acid (referred to as type C18:0) in relation to the composition of total fatty acids in a natural and untreated cocoa butter, in particular the percentage of stearic acid is less than 32%, preferably less than 31.5%; than 31%; than 30.5%; than 30%; than 29.5%; than 29%; than 28.5%; than 28%; than 27.5%, than 27%; than 26.5%, 26%; than 25.5% or than approximately 25%, these percentages by weight being calculated as an absolute value by weight across the totality of the fatty acids in the cocoa butter composition, preferably of the cocoa butter distillate. Initially, the percentage of stearic fatty acid in an untreated, but refined cocoa butter is approximately 36%.

Preferably, the composition of the cocoa butter of the invention is characterised by a preferred ratio between the palmitic fatty acids (of type C16:0) and stearic fatty acids (of type C18:0) and which is comprised between 25% and 33%, preferably between 27% (that is to say equal to or greater than 27%) and 33% by weight for the palmitic fatty acid (of type C16:0) and between 25% and 32% by weight for the stearic fatty acid (of type C18:0).

According to the invention, the composition may preferably comprise 27% or more than 27% by weight of palmitic acid and 32% or less than 32%, 31.1% or less than 31.5%, 31% or less of 31%, 30.5% or less than 30.5%, 30% or less than 30%, 29.5% or less than 29.5%, 28% or less than 29%, 28.5% or less than 28.5%, 28% or less than 28%, 27.5% or less than 27.5%, 27% or less than 27%, 26.5% or less than 26.5%, 26% or less than 26%, 25.5% or less than 25.5%, 25% or less than 25% by weight of stearic acid.

Alternatively, the composition may comprise 28% or more than 28% by weight of palmitic acid and 31% or less than 31%, 30.5% or less than 30.5%, 30% or less than 30%, 29.5% or less than 29.5% 29% or less than 29%, 28.5% or less than 28.5%, 28% or less than 28%, 27.5% or less than 27.5%, 27% or less than 27%, 26.5% or less than 26.5%, 26% or less than 26%, 25.5% or less than 25.5%, 25% or less than 25% by weight of stearic acid.

Then, the composition may comprise 29% or more than 29% by weight of palmitic acid and 30% or less than 30%, 29.5% or less than 29.5%, 29% or less than 29%, 28.5% or less than 28.5%, 28% or less than 28%, 27.5% or less than 27.5%, 27% or less than 27%, 26.5% or less than 26.5%, 26% or less than 26%, 26.5% or less than 25.5%, 25% or less than 25% by weight of stearic acid.

In addition, the composition may comprise 30% or more than 30% by weight of palmitic acid and 29% or less than 29%, 28.5% or less than 28.5%, 28% or less than 28%, 27.5% or less than 27.5%, 27% or less than 27%, 26.5% or less than 26.5%, 26% or less than 26%, 25.5% or less than 25.5%, 25% or less than 25% by weight of stearic acid.

In addition, the composition may comprise 31% [or] more than 31% by weight of palmitic acid and 27% or less than 27%, 26.5% or less than 26.5%, 26% or less than 26%, 25.5% or less than 25.5%, 25% or less than 25% by weight of stearic acid.

Finally, the composition may comprise 32% or more than 32% by weight of palmitic acid and 26% or less than 26%, 25.5% or less than 25.5%, 25% or less than 25% by weight of stearic acid.

Another aspect of the invention relates to a preparation process for obtaining a cocoa butter, preferably having the composition of the cocoa butter of the invention, in particular one or more distillate(s) of a cocoa butter, which is advantageously obtained by molecular distillation of a cocoa butter that is unprocessed, but refined or partially refined, this unprocessed but refined cocoa butter has a quantity of Free Fatty Acids (FFA), expressed in % of oleic acid less than 1.75%, preferably less than 1.5%, or even less than 1%, more particularly between 1.3% and 1.5%, these percentages being calculated by weight in relation to the total weight of cocoa butter, it being possible for these levels to be expressed in mg of KOH necessary in order to neutralise the acidity of 1 gm of fat (Acid Value or Acid Number).

The correspondence between the units is as follows: A level of GLA (or FFA) of 1% expressed as oleic acid is equal to an acid value or number of 1.99 mg of KOH per gm of fat

The molecular distillation process of the invention, makes it possible to generate, from the said unprocessed but refined cocoa butter, one or more distillate(s) (olein) and one or more residue(s) (stearin).

The molecular distillation steps may be carried out either alone or in combination with one or more steps of other fractionation processes performed prior to or after the molecular distillation step(s).

Preferably, the molecular distillation is carried out by a heat treatment process at a temperature that is greater than 250° C., more particularly at a temperature comprised between 260° C. and 320° C. and in which the distillation is carried out by a cooling from a temperature comprised between 250° C. and 320° C., to a temperature that is lower than 50° C.

In the process of the invention, the molecular distillation is carried out at a pressure that is less than 0.10 mbar, preferably between 0.01 mbar and 0.005 mbar.

The process of the invention may also include the additional steps of the preparation of a food composition that includes cocoa butter, in particular chocolate, by the addition of other usual components for the preparation of chocolate of such types as dark chocolate, milk chocolate, or white chocolate, that is to say addition of sugar and optionally cocoa powder and/or a dairy ingredient and/or other additives, before or during the steps of conching and tempering in order to form this food composition, preferably of chocolate.

The term “unprocessed but refined cocoa butter” is understood, in the present invention, to refer to a cocoa butter which is extracted from the bean by a physical process, without having received a chemical treatment such as a transesterification. However, this “unprocessed but refined cocoa butter” is also a cocoa butter that is partially or completely refined. This refining is an entirely physical process and not a chemical one.

According to the invention, “an unprocessed but refined cocoa butter” is extracted from cocoa beans and may comprise from 20% to 26% by weight of palmitic fatty acid (of type C16:0), from 29% to 38% by weight of stearic fatty acid (of type C18:0), from 29% to 38% by weight of oleic fatty acid (of type C18:1), and from 2% to 4% by weight of linoleic fatty acid (of type C18:2) and about 1% by weight of arachidic acid (of type C20:0).

However, these percentages may vary depending on the geographical origin of the cocoa beans: in South America, the percentage by weight of linoleic fatty acid, type C18:2 is greater than 3%; in Asia, the percentage by weight of linoleic fatty acid, type C18:2 is less than 2.5%; and in West Africa, the percentage by weight of linoleic fatty acid, type C18:2 is comprised between 2.5% and 3%.

The invention also relates to the composition of the cocoa butter, in particular the cocoa butter distillate, of the invention obtained directly from the said fractionation process of the invention and which exhibits, at a temperature equal to or less than 20° C., a hardness—preferably expressed in terms of solid content, that is lower than that of untreated but refined cocoa butter, and relates as well to the food or cosmetic composition comprising the said cocoa butter distillate. The other fraction also obtained by the process of the invention is a retentate which advantageously presents a fatty acid profile similar to the refined cocoa butter, from which it is derived and can be used for the same applications as this refined cocoa butter.

According to the invention, the composition of the cocoa butter, preferably the cocoa butter distillate, exhibits a significant modification in the ratio between the totality of the different fatty acids in relation to the unprocessed but refined cocoa butter which is the natural product used as the starting material in the process of the invention, in particular a cocoa butter in which the percentage by weight of palmitic fatty acid (C16:0) increases by at least 3%, preferably by at least 5%, while the percentage by weight of stearic fatty acid (C18:0) decreases by at least 3%, preferably by at least 5%, in relation to the unprocessed, but refined cocoa butter, these percentage being calculated as an absolute value by weight across the totality of the fatty acids in the composition of the cocoa butter.

The present invention also relates to a food composition for humans or animals, that comprises or incorporates the cocoa butter composition of the invention, preferably the distillate of the process of the invention.

In the food composition of the invention, the cocoa butter distillate replaces the olein fat of the palm oil or other solid or liquid fats of animal or plant origin, preferably for a percentage of at least 0.5%, preferably equal to or greater than 5%, 10%, 15%, 20% or 25%, and less than 100% of the quantity of olein fat.

Preferably, the food composition is selected from the group consisting of spreads of the water-in-oil type or of the oil-in-water type, in particular spreads without palm oil, including chocolate spreads, ice creams, ganaches and couverture chocolate, in particular for frozen products, anhydrous fillings, chocolate bars or chocolate drinks.

The present invention also relates to a cosmetic composition comprising the cocoa butter distillate according to the invention and one or more cosmetic active substance(s).

The present invention will be described in detail in the examples here below with reference to the appended figures presented by way of non-limiting illustrations of the invention.

### BRIEF DESCRIPTION OF THE FIGURES

FIG. 1 schematically represents the various different steps of the method of the invention.

FIG. 2 schematically represents an example of a molecular distillation column.

FIG. 3 represents the yield of distillate from a cocoa butter treated by fractionation, as a function of the evaporation temperature.

FIG. 4 represents the melting profile (content of solid fats at different temperatures) determined by NMR (Nuclear Magnetic Resonance) of different fractions obtained by the process of the invention compared to that of untreated cocoa butter.

FIGS. 5 and 6 respectively represent the NMR determined melting profile of the fractions of cocoa butter distillates obtained for different evaporation temperatures at different temperatures referred to as “tempering” temperatures (at 20° C. and 26° C. for FIG. 4, and at 26° C. for FIG. 5).

FIGS. 7 and 8 respectively represent the DSC (differential scanning calorimetry) melting profiles of the fractions of distillates and of cocoa butter residues obtained by the process of the invention for different evaporation temperatures.

FIG. 9 shows the fatty acid profile of the different fractions of the compositions of the invention as a function of the temperatures applied.

### DETAILED DESCRIPTION OF THE INVENTION

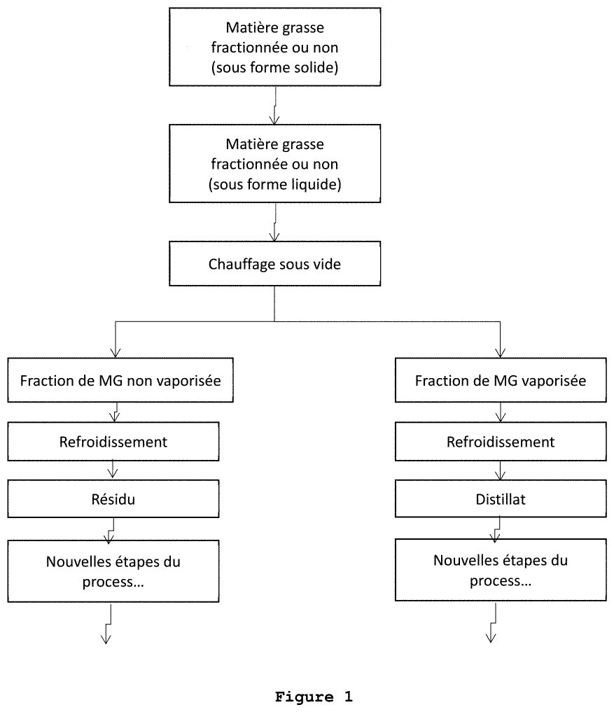

The process according to the invention is characterised by the following steps shown schematically in the appended FIG. 1:
An unprocessed cocoa butter, preferably refined or partially refined, is heated, in which the proportion of free fatty acids (FFA for Free Fatty Acids) is less than 1.75%, in particular less than 1.5%, or close to 1.3%, in a manner such as to render it entirely into liquid form and so as to supply the installation continuously; the cocoa butter is then entrained in the installation under vacuum and is heated indirectly by making use of a thermal fluid, up to a sufficient and necessary temperature, so as to ensure partial evaporation of its constituents;
the vapours thus formed are condensed on the condenser of the installation which is maintained at a temperature that is lower than the evaporation temperature and also higher than the melting point of the fraction collected, in order to ensure the flow thereof. This fraction obtained is described as being the “distillate” or “the olein”;
the cocoa butter that has passed through the evaporator and which has not vaporised, is cooled to a temperature that is lower than the evaporation temperature, but also higher than the melting point of the fraction obtained and described as being the “residue” or “retentate” or “stearin”.

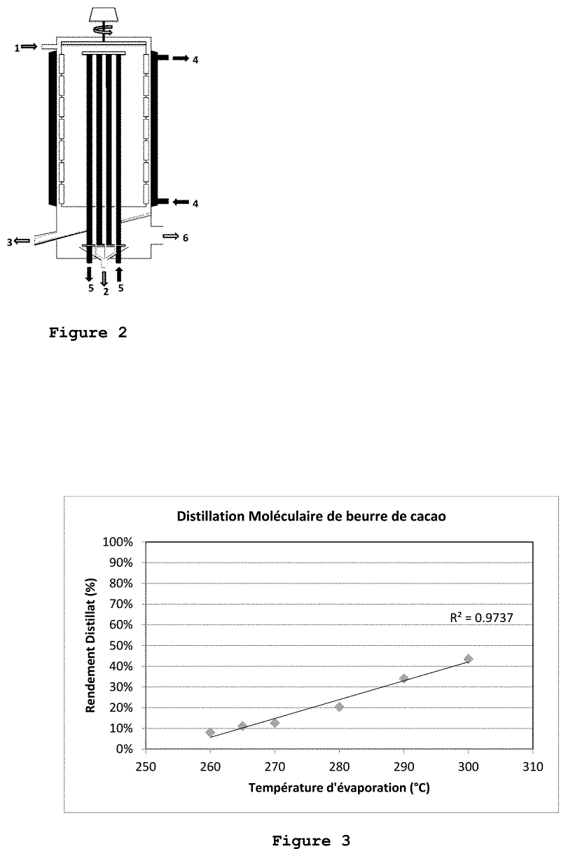

The schematic diagram representing a type of non-exclusive (non-proprietary) molecular distillation column is shown in FIG. 2, as well as in the commercial brochure of the company VTA Verfahrenstechnische Anlagen GmbH & Co. KG, Bernrieder Str. 10 D-94559 Niederwinkling, Germany).

In the schematic diagram of FIG. 2, the reference numeral 1 represents the fat supply; reference 2 represents the obtaining of the distillate; reference 3, obtaining of the residue; reference 4, the heating element; reference 5, the cooling element; and reference 6, the vacuum creating device.

The molecular distillation of cocoa butter makes it possible to obtain two fractions: the distillate or “olein” and the residue or “stearin” in variable proportions depending on the parameters applied.

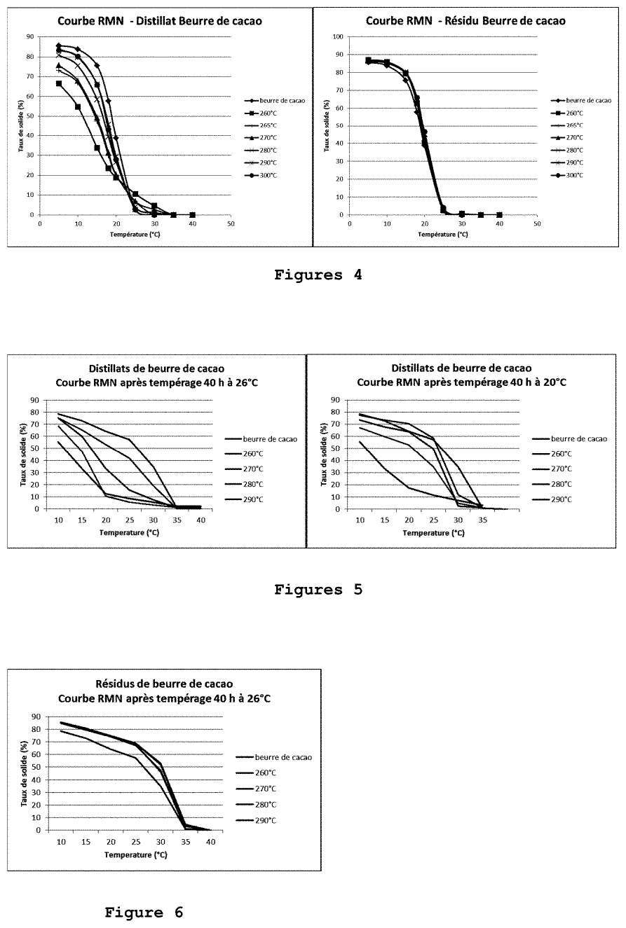

The graph in FIG. 3 illustrates the effect of the temperature of the evaporator on the yield of the distillate and the graphs in FIGS. 4 to 6 present the impact of the evaporation temperature on the quality of the fractions obtained (distillates and residues) characterised by the melting profile.

Consequently, the advantageous particularity of the molecular distillation process implemented is to make it possible in an unexpected manner, to obtain very different distillates from the starting cocoa butter while also recovering one or more residue(s) whose behaviour is very close to that of the untreated cocoa butter.

Indeed, the distillates obtained by the process of the invention in an unexpected and advantageous manner exhibit a texture that is less hard or firm than that of the untreated cocoa butter, with a melting curve that is less steep than that of the cocoa butter, because the distillate obtained at 260° C. presents more solids at 25° C./30° C. and less solids at 20° C. and below.

As a consequence, this distillate is therefore a more heat-tolerant product. The residues obtained in an unexpected manner exhibit a texture that is very close to that of untreated cocoa butter, or even slightly more solid, and can therefore be used under the same conditions as this untreated cocoa butter.

Thus, the two fractions obtained are each independently recoverable and applicable in multiple food or non-food technical fields, in particular in cosmetics.

The NMR curves were also produced after tempering of the products. The so-called “tempering” operation is very important for cocoa butter, because it will provide the ability to obtain a stable and orderly crystallisation of the fat. Commonly, cocoa butter and “solid” fractions are tempered at 26° C., while more “liquid” fractions are tempered at 20° C.

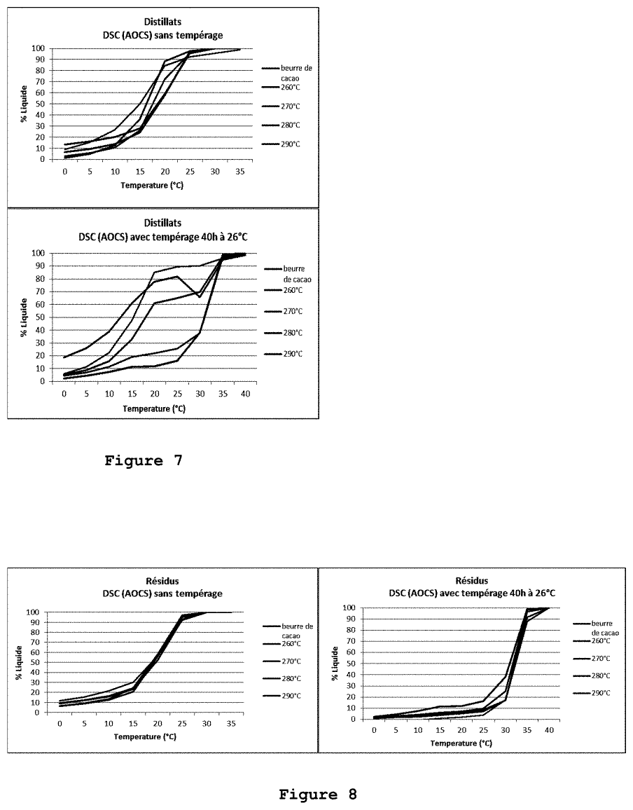

The fractions obtained were also characterised on the basis of other methods of analysis, such as differential scanning calorimetry or DSC as presented in FIGS. 7 and 8 (with and without tempering) and expressed in percentage % of liquid obtained by calculation of the ratios of surface areas (enthalpy).

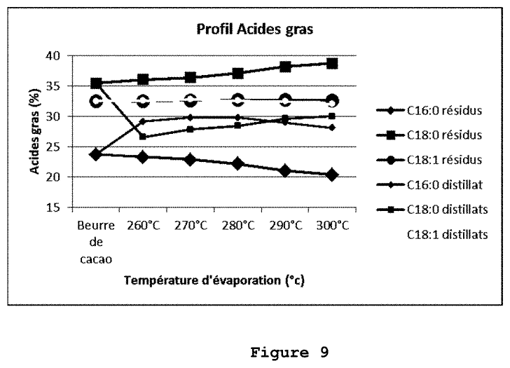

The graph of FIG. 9 presents the typical fatty acid composition of the different fractions obtained by varying a single key variable of the process of the invention: the evaporation temperature.

### Example 1: DSC Measurement with Tempering

Fill a capsule with previously melted fat and place the sample for a period of 10 minutes at 60° C. Then place the sample for a period of 1 hour 30 minutes at 0° C. before placing the sample for a period of 40 hours at the desired temperature (20° C., 26° C., etc).

DSC measurement—AOCS (American Oil Chemists' Society) method:
AOCS Cj 1-94: official method (=86 minutes):
20 to 80° C. (15° C./minute)
80° C. for 10 minutes
80° C. to −60° C. (−10° C./minutes)
−60° C. for 30 minutes
−60° C. to 80° C. (5° C./minutes)

### Example 2: NMR Measurement with Tempering

| | AOCS Cd 16-18 |
|---|---|
| Tubes of samples | |
| External diameter (mm) | 10.0 ± 0.1 |
| Wall thickness (mm) | 0.6 |
| Length (mm) | 150 |
| Material | glass |
| Sample quantity | 4 ± 1 cm |
| Operating conditions | |
| Mixing at temperature (° C.) | 100 |
| 5 to 10 minutes at ° C. | 60 |
| Time at 0° C. (minutes) | 90 |
| Time at 26° C. (hours) | 40 |
| Time at 0° C. (minutes) | 90 |
| Time at 5° C. (minutes) | 60 |
| Time at 10° C. & more (minutes) | 60 |

### Example 3: NMR Measurement without Tempering: (Referred to as Rapid Method)

This method is based on the use of the following material:
An apparatus for Nuclear Magnetic Resonance MINISPEC MQ 20
PYREX tubes measuring 10 mm in diameter;
Three calibration standards with tube 1 (0%), tube 2 (30.2%) and tube 3 (73.8%);
Four cryostats: Julabo at temperatures of 0° C., 5° C., 18° C., 30° C.

The analysis of the sample is carried out by means of the following steps:
Heat the sample in a “water bath”, in an oven or by means of a microwave heating device, in order to ensure that it is completely liquid;
Introduce the sample tested by using a 5 ml syringe, into the Pyrex tube up to the half-way point;
Place the tube in the heating rack for a period of 10 minutes;
Take the tube out of the rack, place it in the vessel containing the liquid nitrogen for a period of 1 minute;
Take the tube out of the vessel and insert it in the rack of the appropriate water bath (0, 5, 18, 30° C.) for a period of 7 minutes;
Take the tube out of the water bath and insert it into the measuring apparatus;
The result read is expressed as a percentage % of solid in the fat. The result measured by the apparatus (value=x) must be calculated according to the following formula: NMR reading Corman (coordinate manipulation and analysis)=SI (x<32.5; x*0.941358025; 30.5+(x−32.4)*0.956834532)

This method differs from the ISO method known to the person skilled in the art for this type of measurement by way of the following elements:
Faster cooling of the sample in liquid nitrogen (ISO=60 min. at 0° C.));
A shorter analysis time (ISO=30 minutes at the appropriate temperature)

### Example 4: Analysis of the Fatty Acid Profile

Determination of the composition of fatty acid methyl esters of the fat by capillary column gas chromatography (CGC).

Materials: Chromatograph equipped with a capillary column with intermediate polarity, a flame ionization detector (FID), a computer with integration software.

Principle of the Method: The fat is esterified in the presence of methanol. The fatty acid methyl esters are separated on an intermediate polar column and are raised according to their molecular weight. The surface area corresponding to each of them is calculated and reported in relation to the total surface area of the various fatty acids in order to obtain a percentage.

1.-20. (canceled)

21. A cocoa butter composition comprising a percentage by weight of palmitic fatty acid (C16:0) that is greater than or equal to 27%, calculated in relation to the total percentage by weight of fatty acids in the cocoa butter composition.

22. The composition according to claim 21, comprising a percentage by weight of palmitic fatty acid (C16:0) that is greater than or equal to 28%, preferably greater than 28.5%, 29%, 29.5%, 30%, 30.5%, 31%, 31.5%, 32%, 32.5% or 33%, calculated in relation to the total percentage by weight of fatty acids in the composition of the cocoa butter.

23. The composition according to claim 21, comprising a percentage by weight of stearic fatty acid (C18:0) that is less than or equal to 32%, calculated in relation to the total percentage by weight of fatty acids in the composition of the cocoa butter.

24. The composition according to claim 23, comprising a percentage by weight of stearic fatty acid (C18:0) that is less than 31%, preferably less than or equal to 29.5%, 29%, 28.5%, 28%, 275%, 27%, 26.5%, 26%, 25.5%, or 25%, calculated in relation to the total percentage by weight of fatty acids in the composition of the cocoa butter.

25. The composition according to the claim 21, comprising between 27% and 32% by weight of palmitic acid (C16:0), and between 32% and 25% by weight of stearic acid (C18:0).

26. The composition according to claim 25 comprising 29% by weight of palmitic acid (C16:0) and 25% by weight of stearic acid (C18:0).

27. The composition according to claim 25, comprising 27% by weight of palmitic acid (C16:0) and 30% by weight of stearic acid (C18:0).

28. A fractionation process for fractionating a refined cocoa butter into one or more distillate(s) and one or more residue(s), by one or more steps of molecular distillation of refined cocoa butter.

29. The process according to claim 28, combined with one or more step(s) of another process of separation by fractionation.

30. The process according to the claim 28, wherein the molecular distillation is carried out by a heat treatment process at a temperature that is greater than 250° C.

31. The process according to claim 29, wherein the distillation is carried out by a heat treatment process at a temperature that is comprised between 260° C. and 320° C.

32. The process according to the claim 28, wherein the distillation is carried out by a cooling from a temperature comprised between 250° C. and 320° C. to a temperature that is lower than 50° C.

33. The process according to the claim 28, in which the distillation is carried out at a pressure that is less than 0.10 mbar.

34. The process according to the claim 28, which includes addition to the cocoa butter distillate of sugar and optionally cocoa powder and/or a dairy ingredient.

35. The process of claim 34, which further comprises the steps of conching and tempering in order to form the chocolate.

36. The process according to claim 28, wherein the one or more distillate(s) presents the composition of cocoa butter comprising a percentage by weight of palmitic fatty acid (C16:0) that is greater than or equal to 27%, calculated in relation to the total percentage by weight of fatty acids in the cocoa butter composition.

37. The process according to claim 28, wherein the one or more distillate(s) present(s) the composition of cocoa butter comprising between 27% and 32% by weight of palmitic acid (C16:0), and between 32% and 25% by weight of stearic acid (C18:0).

38. The cocoa butter distillate obtained from the fractionation process according to the claim 28, which exhibits, at a temperature equal to or less than 20° C., a hardness—expressed in terms of solid content, that is lower than that of refined cocoa butter.

39. A food composition comprising the cocoa butter according to the claim 21 or a cocoa butter distillate which exhibits, at a temperature equal to or less than 20° C., a hardness—expressed in terms of solid content, that is lower than that of refined cocoa butter.

40. The composition according to claim 39, in which the cocoa butter or the cocoa butter distillate replaces the olein fat of the palm oil or other solid or liquid fats of animal or plant origin, whether or not treated in order to modify the composition and the texture, for a percentage of at least 0.5%, preferably for a minimum percentage equal to or greater than 5%, 10%, 15%, 20%, 25%, and at most 100% of the quantity of olein fat.

41. The food composition according to claim 39, selected from the group consisting of spreads of the water-in-oil type or of the oil-in-water type, including chocolate spreads, ice creams, ganaches and couverture chocolate, anhydrous fillings, chocolate bars or chocolate drinks.

42. A cosmetic composition comprising the cocoa butter according to the claim 21 or a cocoa butter distillate which exhibits, at a temperature equal to or less than 20° C., a hardness—expressed in terms of solid content, that is lower than that of refined cocoa butter, and one or more cosmetic active substance(s).
